(19) United States
(12) Patent Application Publication
(10) Pub. No.: US 2004/0214315 A1
(43) Pub. Date: Oct. 28, 2004

(54) Ultrathin-walled multi-well plate for heat block thermocycling

(75) Inventors: Hans-Peter Saluz, Oberbodnitz (DE); Alexandre Tretiakov, Jena (DE)

Correspondence Address: JORDAN AND HAMBURG LLP, 122 EAST 42ND STREET, SUITE 4000, NEW YORK, NY, 10168, US

(73) Assignee: Analytik Jena AG, Jena (DE)

(21) Appl. No.: 10/848,608

(22) Filed: May 17, 2004

Related U.S. Application Data
(63) Continuation of application No. 09/830,511, filed on Apr. 26, 2001, now abandoned.
Application No. 09/830,511 is a 371 of international application No. PCT/EP99/08178, filed on Oct. 28, 1999.

(30) Foreign Application Priority Data
Oct. 29, 1998 (EP) 98120187.4

(51) Int. Cl. C12M 1/02; C12M 1/38

(52) U.S. Cl. 435/303.1; 435/305.2; 435/287.2

(57) ABSTRACT

An ultrathin-walled multiwell reactor for heat block thermocycling of samples includes an array of small-volume wells of identical height with similarly shaped sample wells formed in a top surface of a heat block. The multiwell plates are preferentially vacuum formed out of a 30-50 micron thick thermoplastic film and can be used for rapid, oil-free temperature cycling of small (1-10 μl) volume samples.

This is a continuation application of application Ser. No. 09/830,511 filed Apr. 26, 2001.

### BACKGROUND

The invention relates to plastic plates for conventional heat block thermocycling of biological samples, particularly to multiwell plates. More specifically, it relates to ultrathin-walled multiwell plates with an improved heat transfer to small-volume samples. Such plates can be used for rapid temperature cycling of multiple, small-volume samples (i.e. 1-20 μl) by using heat block thermocyclers with an increased block temperature ramping rate (i.e. 4° C./second and greater) and standard heated-lid technology for sealing the samples.

Temperature cycling of biological samples is a central moment in DNA amplification by the polymerase chain reaction (PCR) (Saiki et al., Science, 239, 487-491, (1988)). Much effort is being expended in developing various alternative reactors and technologies for rapid temperature cycling of small-volume samples (Kopp et al., Science 280, 1046-1048, (1998); Belgrader et al., J. Forensic Science 43, 315-319, (1998); Wittwer et al., Analytical Biochem., 186, 328-331 (1990) and U.S. Pat. No. 5,455,175; Woolley et al., Analytical Chem., 68, 4081-4086 ((1996)).

One commercially available type of microreactor and thermocycler for rapid temperature cycling of small samples is a glass capillary tube and a hot-air thermocycler from Roche Molecular Biochemicals (cat No. 1909 339 and cat No. 2011468, respectively). The glass capillary tube can hold reaction volumes ranging from 10 to 20 μl. The hot-air thermocycler can hold 32 capillaries and perform 30-40 PCR cycles in 20-30 minutes. However, this rapid DNA amplification technology has various disadvantages, for example:

a) The handling of the individual capillaries is relatively cumbersome.

b) The relatively large glass surface adsorbs components of the standard PCR-mixtures. This might inactivate the reaction. Therefore, various carrier molecules, i.e. proteins or even DNA, must be added and the concentrations of the components reoptimized.

c) The cost of the capillary tube, as a disposable PCR container, is high when compared to the standard 0.2 ml PCR tube.

d) The experimental throughput using this system is limited.

It is surprising that only little research has been conducted to improve the basic performance in sample size and speed of the widely used, conventional heat block thermocycling of samples contained in plastic tubes or multiwell plates.

One known improvement of heat block temperature cycling of samples contained in plastic tubes has been described by Half et al. (Biotechniques, 10, 106-112, (1991) and U.S. Pat. No. 5,475,610). They describe a special PCR reaction-compatible one-piece plastic microcentrifuge tube, i.e. a thin-walled PCR tube. The tube has a cylindrically shaped upper wall section, a relatively thin (i.e. approximately 0.3 mm) comically-shaped lower wall section and a dome-shaped bottom. The samples as small as 20 μl are placed into the tubes, the tubes are closed by deformable, gas-tight caps and positioned into similarly shaped conical wells machined in the body of the heat block. The heated cover to compresses each cap and forces each tube down firmly into its own well. The heated platen (i.e. heated lid) serves several goals by supplying the appropriate pressure to the caps of the tubes: it maintains the conically shaped walls in close thermal contact with the body of the block; it prevents the opening of the caps by increased air pressure arising in the tubes at elevated temperatures. In addition, it maintains the parts of the tubes that project above the top surface of the block at 95°-100° C. in order to prevent water condensation and sample loss in the course of thermocycling. This makes it possible to exclude the placing of mineral oil or glycerol into the wells of the block in order to improve the heat transfer to the tubes and the overlaying of the samples by mineral oil that prevented evaporation but also served as added thermal mass. In addition, the PCR tubes can be put in a two-piece holder (U.S. Pat. No. 5,710,381) of an 8×12, 96-well microplate format, which can be used to support the high sample throughput needs with any number between 1 and 96 individual reaction tubes.

In DE 4022792 the inventors describe a plate with cylindrically shaped walls of the wells and spherically shaped bottoms thereof. The individual wells of the plate were formed by melting a polycarbonate sheet in the range of 0.27-0.5 mm by a stream of hot air. This technology leads to relatively thin walls in the range of 0.08-0.2 mm. The biological samples were placed into the wells, covered with polycarbonate film (0.1 mm) and the individual wells were thermosealed by a special press. Upon sealing the plate was placed on the thermoblock and fixed by screws. Though theoretically the heat transfer to 30 the samples is improved, however, the way of positioning the plate on the block and the cylindrical and spherical geometry of the well prevent a close thermal contact with the heating block. During thermocyling, due to the large thermal expansion, the plate fixed by screws becomes deformed and the close thermal contact is not maintained anymore. Therefore, by using the above technology rapid cycling reactions cannot be performed.

Another known improvement of heat block thermocycling is described in PCT patent application WO 98/43740 and concerns a heat block thermocycler with an increased ramping rate, i.e. 4° C./second. The thermocycler can hold 96 PCR tubes (each of a volume of 0.2 ml) or 96-well PCR plates. Theoretically, the thermocycler can perform 30 PCR cycles in 20-30 minutes, provided that only a few seconds are spent to reach the temperature equilibrium between the heat block and the samples.

However, as described in U.S. Pat. No. 5,508,197, even if the temperature of the heat-transfer media, i.e. water, is changed almost instantaneously, it takes approximately 15 seconds to reach equilibrium between water and the 15-20 μl samples in the standard PCR plates. This means that for 30 PCR cycles approximately 20 minutes are spent to reach the equilibrium between heat-transfer media and the 15-20 μl samples in the plates.

In comparison, the above mentioned heat block cycler (WO 98/43740) operating at a ramping rate of 4° C./second, needs for the heat-block temperature transitions during 30 PCR cycles 10 minutes only. This shows that the major limiting factor for rapid temperature cycling of small samples in plastic PCR tubes or PCR plates is the low efficiency of the heat transfer through the walls of conventional PCR tubes or plates, respectively.

### SUMMARY

The present invention concerns plastic multiwell plates for performing heat block thermocycling of multiple samples. More specifically, it concerns ultrathin-walled multiwell plates with an improved heat transfer to small samples. Ultrathin-walled multiwell plates are suited for rapid, oil-free, heat block temperature cycling of small volume samples (i.e. approximately 1-20 μl), whereas the lower limit is given by the reliability of the conventional pipetting systems.

### BRIEF DESCRIPTION OF THE DRAWINGS

FIG. 1a is a plan view of an embodiment of a multiwell plate according to the present invention.

FIG. 1b is a cross-sectional side elevation view of the multiwell plate of FIG. 1a.

FIG. 2 is a cross-sectional side elevation view of the multiwell plate of FIG. 1a positioned in a block of a thermal cycler.

FIG. 3 is a cross-sectional view of the multiwell plate without a support frame attached.

FIG. 4 is a cross-sectional view of a support frame having an array of holes for accepting the multi-well plate of FIG. 3.

FIG. 5 is perspective view of the support frame of FIG. 4.

### DETAILED DESCRIPTION

One aspect of the present invention concerns a multiwell plate having a considerably decreased thickness (i.e. approximately 7.5-15 fold) of well walls when compared to known thin-walled PCR tubes (U.S. Pat. No. 5,475,610). This can be achieved, for example, by means of thermoforming the multiwell plates out of thin thermoplastic films. Such thermoplastic films are, for example, polyolefin films, such as metallocene-catalyzed polyolefin films and/or copolymer films. Usually, the multiwell plate is vacuumformed out of cast, unoriented polypropylene film, polypropylene-polyethylene copolymer films or metallocenecatalyzed polypropylene films. The film is formed into a negative (“female”) mould comprising a plurality of spaced-apart, conically shaped wells which are machined in the body of a mould in the shape of rectangular- or square-array. The thickness of the film for vacuumforming conically shaped wells is chosen according to the standard rule used for thermoforming, i.e. thickness of the film=well draw ratio×thickness of the wall of the formed well.

For example, vacuumforming wells with a draw ratio of two and an average thickness of the walls of 30 microns requires a film thickness of 60 microns. The average optimum wall thickness was found to be 20-40 microns. The thickness of the well is reduced 7.5-15 fold when compared to the wall thickness of the formerly improved PCR tube described in U.S. Pat. No. 5,475,610. Using the Fourier equation for heat transfer and the equation for temperature transfer through solid substances, it can be shown that heat transfer through one square millimeter of the surface of the well of the plate is increased 7.5-15 fold and the time of temperature transfer through the wall is decreased 56-225 fold when compared to the PCR tube. This drastic decrease in time can be explained by the fact that the time needed for the transfer of temperature front is proportional to the square power of distance. It can be easily calculated that the time of the temperature transfer through the ultrathin walls of the multi-well plate is in the range of milliseconds, whereas for the PCR tube (U.S. Pat. No. 5,475,610) it is in the range of seconds. This explains the well known fact that thin (20-40 microns) plastic films are poor theremo insulators.

The thickness of the walls of the formed wells is gradually reduced to the bottoms of the wells due to vacuumforming of the wells into a negative mould. This geometry of the walls of the wells provides several advantages:

The relatively thick upper parts of the walls of the wells cause additional rigidity of the whole multiwell plate. During heating of the heat block of the thermocycler, a vertical temperature gradient is formed in the sample, due to the gradient of the well-wall thickness. This vertical temperature gradient causes intensive convective mixing of the sample in conically shaped wells and increases the heat transfer through the sample. In comparison, this convective mixing of the sample is much less efficient in conventional PCR plates/tubes with a uniform wall thickness.

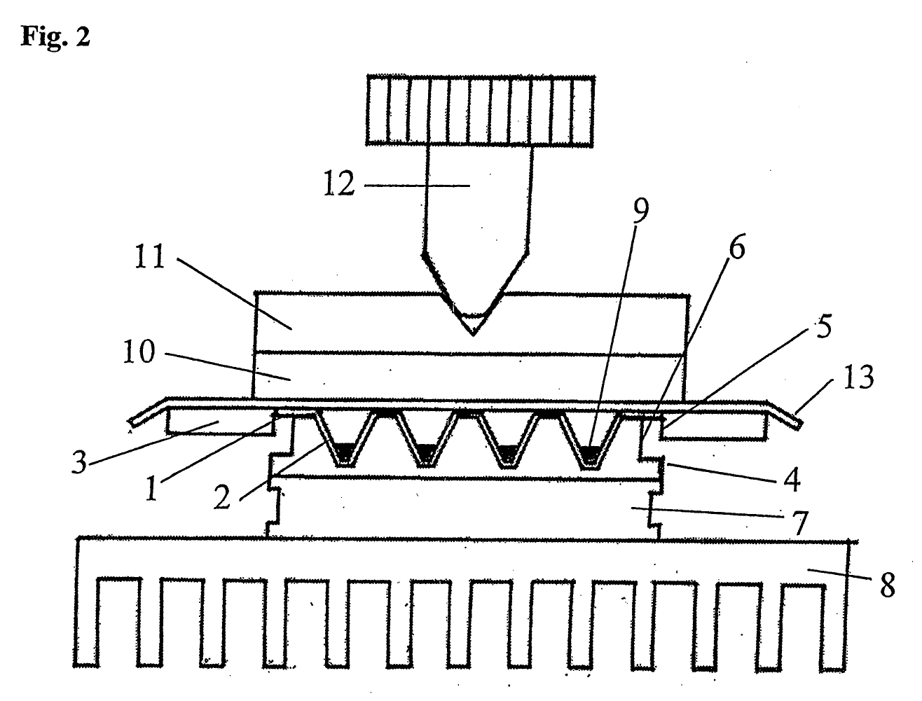

Another aspect of the invention concerns the height of the wells of the multiwell plate. The height of the conically shaped wells is equal to the height of the similarly shaped sample wells machined in the body of the heat block. Thus, this geometry of the wells (2) enables the positioning of the plate (1) on the heat block (4) as shown in FIG. 2. As shown (FIG. 2), in contrast to the conventional PCR plates, the walls of the wells (2) of the multi-well plate (1) do not project above the top surface of the block (4). The type of positioning provides several advantages: The pressure caused by the screw (12) to the lid (10) (heating element (11)) can be increased in order to obtain efficient sealing of the samples (9) sealed, for example, by a silicon mat (13). In this case the pressure is to actually directed to those parts of the multiwell plate (1) which are supported by the top surface of the heat block (4) (or by parts of the top surface surrounding individual wells depending on the geometry of the heat block) and not to the thin walls of the wells of the plate as it is the case for the PCR tubes or conventional PCR plates. This advantage makes it possibe to increase the sealing pressure of the heated lid (10) several fold when compared to the conventionally used pressure of 30-50 g per well without cracking the conically shaped walls of the wells (2).

The extremely thin walls of the wells, i.e. 20-40 microns, are highly flexible as the multiwell plates are thermoformed out of highly elastic films (or sheets depending on the draw ratio). The walls of the wells are highly resistant against stress cracking, due to their flexibility and elasticity. As the wells of the plate, positioned on the heat block, are tightly sealed at room temperature, the air pressure in the wells will increase at elevated temperatures. The increased air pressure causes a deformation of the walls of wells and brings them in tight thermal contact with the surface of the walls of the individual sample wells machined in the body of the heat block. Standard PCR plates (having relatively thick and rigid walls of the wells) require that the conically shaped walls of the wells have to match perfectly with the shape of the wells machined in the body of the heat block to guarantee a close thermal contact (see for example U.S. Pat. No. 5,475,610). This requirement is not as critical for the ultrathin walled multiwell plates of the invention, due to flexibility and elasticity of the walls of the wells. Using this advantage, special shapes of both, the walls of the wells of the plate and the wells of the heat block can be differently designed. These differently designed wells can promote an even closer thermal contact after positioning the plate into the heat block.

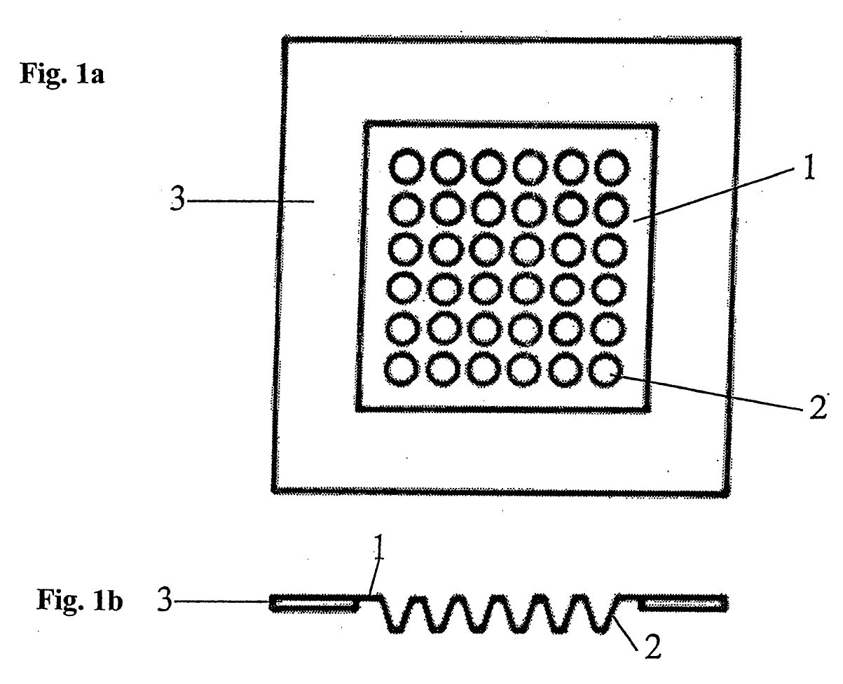

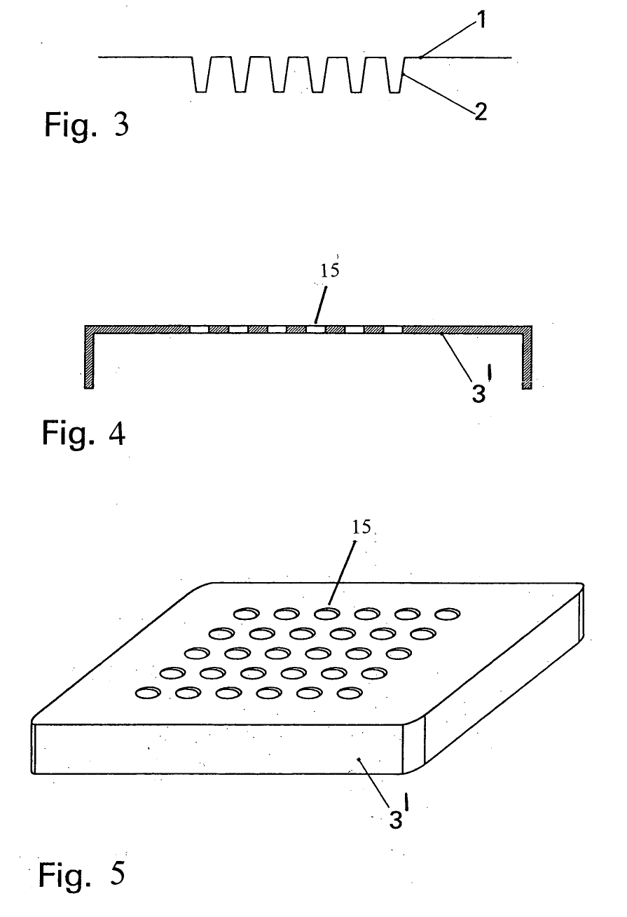

Referring to FIGS. 3-5, another aspect of the invention concerns a support frame 3′ for a multiwell plate 1. As the plates 1 can be formed of very thin films (depending on the draw ratio of the well; supra) the flexibility of, for example, standard-format plates, i.e. 96-well PCR (8,5×12,5 cm) plates, is such that handling is not easily possible anymore. Therefore, depending on the geometry of the plate 1, a supporting frame might be needed, for example for industry standard formats, i.e. 96-, 192-, 384-well PCR plates. This frame can support, for example in case of small plates, the edges of the plate as shown in the form of a support frame 3 in FIGS. 1a and 1b, or individual wells of the plate, or groups of wells. For handling with robots, for example, the frame 3′ of FIGS. 3-5 can be injection molded in the form of the standard skirted microplates containing an array of holes 15 in a top surface of the frame 3′ matching the array of wells of the ultrathin multiwell plate 1. The plate 1 can be attached to the frame 3′ by, for example, heat bonding. However, small format plates including a frame can be formed as a single piece by using specially designed moulds.

The polypropylene-based plastics are PCR-compatible and therefore widely used for injection molding of PCR tubes and/or multiwell plates. In addition, they are resistant to stress cracking and have a reduced water vapor sorption when compared to other plastics (e.g. polycarbonate). Such plates can be thermoformed in both, standard industry formats, i.e. 96-, 192 and 384-well PCR plates for large scale applications, supported by robots and small foot-print formats to match small foot-print thermocyclers, i.e. “personal thermocyclers”.

The following example serves to illustrate the invention but should not be construed as a limitation thereof.

### EXAMPLE

FIG. 1 illustrates a 36-well ultrathin walled multiwell plate according to the invention. The plate was designed for rapid temperature cycling of samples ranging from 0.5-4 μl using a small foot-print pettier-driven heat block thermocycler supplied with a “wine-press” type heated lid (FIG. 2). The volume of the wells is 16 μl and the distance between the wells is 4.5 mm, i.e. industry standard for high sample density 384-well PCR plates. The diameter of the openings of the wells is 3.8 mm and the height of the wells is 3 mm. The average thickness of the walls of the wells is 30 μm. The frame (3) was cut out of a polypropylene sheet of a thickness of 0.5 mm and heat bonded to the plate (1). The area of the plate (1) is 30×30 mm. As shown in FIG. 1, the handling of the plate (1) containing the multiple wells (2) is facilitated, by a rigid 0.5-1 mm thick plastic frame (3) which is heat bonded to the plate. As shown in FIG. 2, the frame (3) is not in direct thermal contact with the block (4) during thermocycling because the inner contour (5) of the frame (3) matches the outer contour (6) of the heat block (4) of the thermocycler (7=thermoelectric heat pump and 8=air-forced heat sink).

The ultrathin walled multiwell plate according to the invention (FIG. 1) was experimentally tested for the amplification of a 455-base pairs long fragment of human papilloma virus DNA. The sample volume was 3 μl. For various PCR reactions, the average ramping rate of the thermo cycler was varied from 4° C. to 8° C. per second. The samples (i.e. standard PCR-mixtures without any carrier molecules) were transferred into the wells of the plate by means of conventional pipetting equipment. The plate was covered by standard sealing film (Microseal A; M J-Research, USA), transferred into the heatblock of the thermocycler and tightly sealed by the heated lid as shown in FIG. 2. Upon sealing, a number of 30 PCR cycles was performed in 15-25 minutes depending on the ramping rate of the thermo cycler. The PCR product was analyzed by conventional agarose electrophoresis. The 455-base pairs long DNA fragment was amplified with a high specificity at the indicated ramping rates (supra).

Plates according to the invention with well volumes of 35 μl were successfully tested for temperature cycling of samples of a volume of 20 μl. Thereby, 30 PCR cycles were performed in 20-30 minutes at a ramping rate of 6° C. per second. Surprisingly, although the average thickness of the walls was 20 microns and the volume of the wells was 35 μl, samples of a volume of as few as 0.5 μl can be easily amplified without reducing the PCR efficiency.

In conclusion, the ultrathin walled multiwell plates according to the invention, allow a simple and rapid loading of multiple samples by conventional pipettes, rapid sealing of all samples by using conventional sealing films and rapid DNA amplification (15-30 minutes for 30 cycles) with an improved specificity typical for rapid cycling (Wittwer et al., Analytical Biochem., 186, 328-331 (1990)) using appropriate heat block thermocyclers (i.e. ramping rate in the range of 4° C. to 8° C. per second).

What is claimed is:

1. An ultrathin-walled multiwell plate for carrying samples for PCR in a heat block for heat block thermocycling of samples comprising:
a plate formed of a thin flexible film and having an array of wells of identical height and shaped for fitting into wells in a top surface of the heat block of the thermocycler, wherein the walls of the wells have an average thickness of 20-40 microns; and
a rigid frame fixed to the plate configured to provide support for said plate when outside of the heat block, the rigid frame containing an array of holes in a top surface matching the array of wells.

2. The ultrathin-walled multiwell plate according to claim 1, wherein the walls of the wells are conically shaped.

3. The ultrathin-walled multiwell plate according to claim 1, wherein the thickness of the walls of the wells decreases from top to bottom.

4. The ultrathin-walled multiwell plate according to claim 1, wherein the wells of said multiwell plate are thermoformed into negative mould.

5. The ultrathin-walled multiwell plate according to claim 1, wherein the walls of the wells are deformable.

6. The ultrathin-walled multiwell plate according to claim 1, wherein the volume of the well is in the range of 16-85 μl.

7. The ultrathin-walled multiwell plate according to claim 1, wherein the rigid frame is formed of a polypropylene sheet of thickness of 0.5 mm and heat bonded to the plate.

8. The ultrathin-walled multiwell plate according to claim 1, wherein the rigid frame is a rigid 0.5-1 mm thick plastic frame heat bonded to the plate.

9. The ultrathin-walled multiwell plate according to claim 1, wherein the rigid frame is below the plate and the array of wells extends through the array of holes.

10. A PCR device comprising;
a plate formed of a thin flexible film and having an array of plate wells having a wall thickness in the range of 20-40 microns;
a rigid frame fixed to the plate and configured to provide support for said plate permitting transport of said plate;
a heat block having heat block wells accepting said array of plate wells of said plate, said heat block wells of said heat block having apertureless bottoms; and
said plate wells having side walls equal in height to said heat block wells.

11. The PCR device according to claim 10, wherein the walls of the plate wells are conically shaped.

12. The PCR device according to claim 10, wherein the thickness of the walls of the plate wells decreases from top to bottom.

13. The PCR device according to claim 10, wherein the wells of said plate are thermoformed into negative mould.

14. The PCR device according to claim 10, wherein the walls of the plate wells are deformable.

15. The PCR device according to claim 10, wherein the volume of the plate wells is in the range of 16-85 μl.
